(19) United States
(12) Patent Application Publication
(10) Pub. No.: US 2019/0227047 A1
(43) Pub. Date: Jul. 25, 2019

(54) CONDITION DIAGNOSIS AND A LIFE-EXTENDING TREATMENT METHOD FOR AN ELECTRIC APPARATUS CONTAINING INSULATION OIL AND ITS ASSOCIATED SYSTEM

(71) Applicant: VP DIAGNOSE AS, SKJEBERG (NO)

(72) Inventor: Egil RAVNEMYR, Gamle Fredrikstad (NO)

(73) Assignee: VP DIAGNOSE AS, SKJEBERG (NO)

(21) Appl. No.: 15/876,898

(22) Filed: Jan. 22, 2018

(51) Int. Cl. G01N 33/28; H01B 3/24; C07F 9/50; G01N 30/20; G01N 30/02

(52) U.S. Cl. CPC G01N 33/287; H01B 3/24; G01N 2030/025; G01N 30/20; C07F 9/5022

(57) ABSTRACT

A condition diagnosis and a life-extending treatment method for an electric apparatus comprising insulation oil and a system to perform the method and comprises addition of a specific group, phospines, of chemical substances to protect metals in transformers and other insulating oil filled apparatuses from sulphidation caused by elemental sulphur.

### FIELD OF THE INVENTION

Method for removal of elemental sulfur from transformer/electrical insulation oils operating in strong electrical and magnetic fields.

### BACKGROUND

During the latest decades, sulfidation has appeared as a problem in electrical oil insulated apparatuses such as transformers, reactors, breakers, and auxiliary devices like e.g. tap changers. In short; anything containing electrical insulation oils and which operates in strong electrical and/or magnetic fields.

The main uses for these oils in transformers are electrical insulation and cooling i.e. to remove heat dissipated from hot surfaces inside the device. For breakers an additional oil function is that of disrupting/quenching the arc that develop during the breaking action.

The sulfidation has been reported to cause disrupted operation and in some cases even costly failures. The failure cause has been and to some extent continue to be “unknown” even by professionals. From the chemical point of view, any molecule/compound that contain one or more sulfur atoms has the potential to become a sulfur source for sulfidation processes.

However, it has become known that certain additives have been used by some insulation oils manufacturing industries, merely for the purpose of boosting their oil product performance in testing methods as defined by International Standard's requirements. It has lead to a change in the International Standards so that now, no additives are allowable, unless they are declared and accepted by the purchaser/user.
However, under certain conditions these additives remain a potential problem in transformers in operation and sometimes the owner decides to “regenerate oil” i.e. remove polar molecules e.g. molecules formed due to oxidation or other degradation processes which appear as a consequence of the chemical reaction conditions inside a transformer.

Some of these processes reuse the polar compound adsorption material (usually Fullers Earth) by a reactivation process where adsorbed hydrocarbons are removed by different processes. In instances when this reactivation process is not completed, the adsorption material may produce/release sulfur in a corrosive form.

It has also been found (by the patent applicant) that elemental sulfur is found in transformers in normal operation and thus is a consequence of not defined processes inside the transformers.

There is an obvious need to inhibit the function of elemental sulfur in order to prevent metal sulfidation (predominantly silver and copper sulfidation).

### SHORT SUMMARY OF THE INVENTION

The invention concerns the use of active phosphines with high selectivity to react with atoms in the chalcogen group such as Oxygen and Sulfur and is an in-situ method for removal of elemental sulfur from electrical insulation oil in an oil filled apparatus comprising, to determine the level of elemental sulfur in the electrical insulation oil and then slowly add highly concentrated phosphine product to the oil. The phosphine product react with the elemental sulfur and form a stable product thus preventing the elemental sulfur from causing metal sulfidation and preventing possible damage and breakdown of electrical oil insulated apparatuses.

In an embodiment of the invention the phosphine product is added in an amount 4 -5 times higher than the elemental sulfur content in the oil. This is advantageous, as this forms a buffer for reacting with more elemental sulfur “leaking out” from winding cellulose and other porous parts of the transformer interior.

In an embodiment of the invention the phosphine product added is Triphenyl-phosphine (TPP), favorable due to due to the large and “dense” appearance of the phenyl group which may create a major increase in selectivity towards atoms such as sulfur and oxygen.

The purpose of the invention is to provide a cost efficient alternative method for the apparatuses owners in combination with providing a significant environmental advantage.

### FIGURE CAPTION

FIG. 1 is a principle sketch of an embodiment of the system to perform one or more embodiments of the method according to the invention.

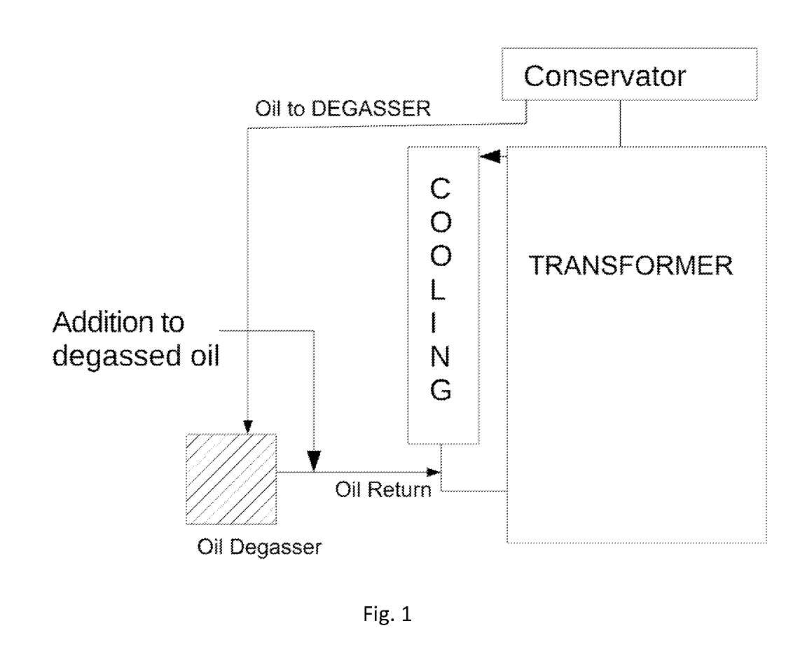

FIG. 2 shows a chromatogram illustrating the ease of determining the presence of TPP, TPPO (Triphenylphosphine oxide) and TPPS by use of a dedicated detector. The chromatogram has been obtained in laboratory testing to determine the selectivity of the reaction when using sulfur free standard base oil for insulation oil manufacturing purposes.

### EMBODIMENTS OF THE INVENTION

The invention concerns the use of active phosphines with high selectivity to react with atoms in the chalcogen group such as Oxygen and Sulfur.

Insulating oils contain a large variety of sulfur containing hydrocarbons. The better refined the oil is, the less sulfur it will contain. There is a general risk that metals that get in contact with oil may be caused to sulphidize i.e. corrode under certain conditions. Sulfur induced corrosion is revealed by a dark, brown or more commonly black, deposition on the metal and on/in the surrounding solid insulation materials e.g. cellulose. This will reduce the insulating ability of the cellulose and the risk for electron migration will increase.

Elemental sulfur exists mainly in three forms including as cyclic molecules having six, seven and most commonly (i.e. most stable) eight sulfur atoms (S₆, S₇ and S₈). Triphenylphosphine (C₁₈H₁₅P) with CAS number 603-35-0, hereinafter referred to as TPP, is an organophosphorus compound with molecular weight 262.29 g/mol. The compound appearance is as white powder, which is solid at standard temperature and pressure. TPP react immediately i.e. very quickly form TPPS with sulfur atoms from S₆, S₇ or S₈ molecules i.e. with elemental sulfur. The only reaction product is triphenylephosphinsulphide (abbreviated TPPS). One molecule TPP bind one sulfur atom and consequently eight molecules of TPP is consumed by one S₈ molecule.

Insulating oil, that at corrosion tests have shown that they contain elemental sulfur i.e. corrosive sulfur, have been treated with TPP. The corrosion test following the TPP-addition indicates no further presence of elemental sulfur. The effect has been statistically confirmed through multiples samples and with real transformers.

Through the addition of TPP to the oil, the problems related to sulfur corrosion are completely eliminated. Elemental sulfur formation has proven to be a very common consequence of oil regeneration. Moreover, it is concluded that TPPS is a very stable and thus a non-corrosion-creating sulphide even at extremely high transformer oil temperatures.

The phosphine molecule may be of different chemical composition but due to its wide spread use in different chemistry related industrial manufacturing processes, TPP (Tri-Phenyl-Phosphine) is suitable to use. The phenyl groups may be substituted with any hydrocarbon group to form a phosphine molecule for use with the present method, but there is an advantage for TPP due to the large and “dense” appearance of the phenyl group. An embodiment of the invention, where TPP is used will then be favorable due to this large and “dense” appearance of the phenyl group, which may create a major increase in selectivity towards atoms such as sulfur and oxygen.

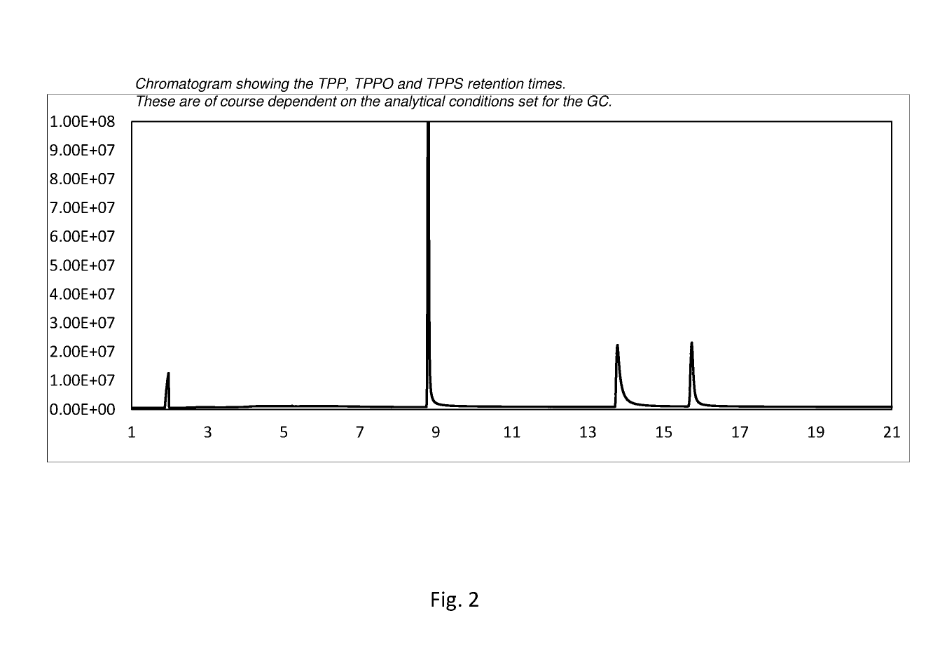

In the chromatogram presented in FIG. 2, the ease of determining the presence of TPP, TPPO (Triphenylphosphine oxide) and TPPS by use of a dedicated detector is illustrated. The chromatogram has been obtained in laboratory testing to determine the selectivity of the reaction when using sulfur free standard base oil for insulation oil manufacturing purposes. No other reactions involving TPP are seen (the GC run was 21 minutes).

In order to determine the TPP ability to react with sulfur even at room temperature (22° Celsius), TPP and S was mixed and analyzed using High Pressure Liquid Chromatography (HPLC). In HPLC the sample is not subjected to elevated temperature when injected and thus the results will ascertain the no reaction has taken place in the injector (In gas chromatography the injector is normally heated to temperatures above 150° Celsius resulting in possible misinterpretations).

The conclusion of that investigation was that sulfur and oxygen compete to react with TPP. This opens up for different methods to apply treatment:
1. The oil is degassed prior to the introduction of TPP. TPP is then added either in powder form or in the form of a concentrated hydrocarbon based solution of TPP. The purpose of degassing is to decrease the oxygen reaction with TPP in favor of the sulfur reaction. It is a costlier method of addition but is also a more sulfur directed method. It will also create a larger “buffer” inside the unit where TPP remain and may be consumed later on as more elemental sulfur is “leaking out” from winding cellulose and other porous parts.
2. It is added, either as powder or in a concentrated hydrocarbon based solution but with no prior degassing. The method will require less preparation and should be performed on a unit where the oil is warm to enable quick reaction.
3. In a cold unit with circulation going, the addition of concentrated hydrocarbon based TPP solution is preferred because TPP solubility time is dependent on absolute temperature and powder may result in particles entering the electrical field.
   Any combination of these methods is possible.

### Reactions

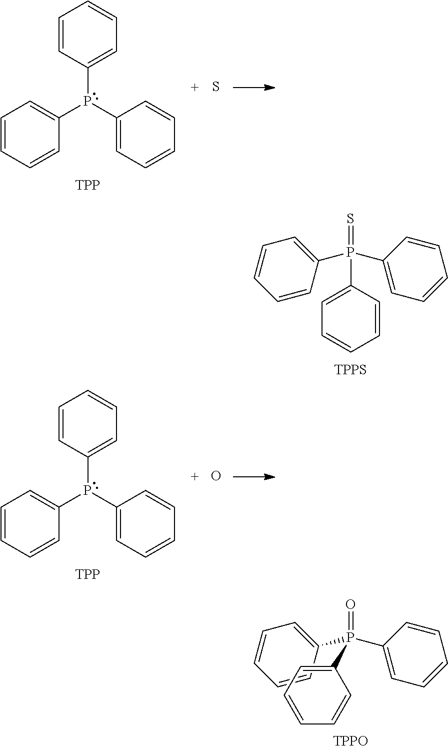

How this Invention is Applied and what is Gained.

The invention is an ad hoc use of TPP or other phosphines of similar reactivity and selectivity for inhibiting the corrosive effects of elemental sulfur or sulfur containing chemical species which easily form elemental sulfur.

The invention creates a possibility to counter-act corrosive behavior experienced when testing properties of certain insulating or base oils. The sulfidation reactions are inhibited with an almost 100% efficiency due to that the reactive sulfur is transformed into a non-reactive variety namely TPPS (if the phosphine selected is TPP).

Laboratory testing has shown that oils that under standardized test conditions produced a “non-corrosive” result after TPP addition, on both silver and copper. Treated oils have been tested also for Breakdown Voltage and Tan delta (loss factor at 90° Celsius) and no negative effects have been found. This is also the expected result due to the very low level of additive used.

In an embodiment of the invention the treatment procedure is as follows:
a)-sampling said electrical insulation oil of said oil filled apparatus for the purpose of
b)-determining elemental sulfur concentration in said electrical insulation oil,
c)-if elemental sulfur is present in said insulation oil, a sulfidation potential exists, then
d)-slowly add a desired amount of a phosphine product to said electrical insulation oil.
   Then add to the insulation oil, at least a stoichiometric, phosphine:sulfur, amount of the phosphine compound.

It is an advantage to add more than the stoichiometric amount of phosphine compound in order to establish a chemical buffer that remain after the treatment has been finished. In an embodiment one may select a treatment level of 4-5 times this concentration.

In an embodiment of the invention one will perform an analysis of the insulation oil, determining qualitatively the presence of elemental sulfur. If the insulation oil contains elemental sulfur a desired amount of phosphine product is added to the insulation oil. The amount of phosphine product to be added, may be selected based on experience figures for similar apparatus and installations.

In an embodiment of the invention the gases present in the transformer oil are removed prior to addition of phosphine product in order to decrease the competing TPPO formation reaction.

Use e.g. a dosing pump to slowly add the liquid containing highly concentrated TPP or other selected phosphine product. The ideal addition time is 24 hours but for practical reasons shorter times can be applied.
Sample from incoming flow to degasser to determine obtained TPP and TPPS concentration.
Sample one week, one month and 6 months later to determine TPP, TPPS and TPPO to evaluate whether there is a need for a further addition of phosphine or if a buffer capacity still exists. One may then consider if it is beneficial to add more phosphide product. The gain for transformer and other machine owners is that no expensive treatments are required and that oil change can be avoided. Consequently this is an environmentally friendly treatment method.

Embodiments of the invention also covers the following:
1. The ad hoc use of TPP or other phosphines of similar reactivity and selectivity for inhibiting the corrosive effects of elemental sulfur or sulfur containing chemical species which easily form elemental sulfur.
2. In situ and in energized condition application of TPP. The inhibition of metal sulfidation-corrosion specifically from elemental sulfur or compounds which may change chemical form into elemental sulfur with sufficient ease to be formed during the operation of a transformer, reactor or other types of devices which have a electrical insulation system consisting of insulating oil containing sulfur in any form and a polymeric solid insulation.
3. Ex situ and in non-energized condition application of TPP. The inhibition of metal corrosion specifically from elemental sulfur or compounds which transform into elemental sulfur with sufficient ease to be formed during the operation of a transformer, reactor or other types of devices which have a winding electrical insulation system consisting of sulfur containing insulating oil and a polymeric solid insulation.
4. The process of adding TPP to the oil by TPP solution injection where the competing reaction with oxygen is kept low by first degassing and heat the oil prior to TPP exposure.
5. The use of TPP after finishing oil maintenance services such as oil regeneration, reclamation or other types of services performed on oils that have been in service in transformers. The analysis is generally performed at a laboratory off site but in an embodiment of the invention all method steps may be performed on site.

The invention is also a system for condition diagnosis and removal of elemental sulfur from electrical insulation oil in an oil filled apparatus comprising
a)-a circulation circuit for said electrical insulation oil
b)-a sampler, at a sampler point, in fluid communication to said electrical insulation oil circuit for sampling said electrical insulation oil of said oil filled apparatus,
c)-an analyzing apparatus for determining concentration of elemental sulfur in said electrical insulation oil,
d)-an injection device, such as an oil pump, at an injection point, in fluid communication for slowly injection of a desired amount of a phosphine product to said electrical insulation oil.

In an embodiment the system comprises a degasser in fluid communication with said electrical insulation oil circuit, for removal of gases present in said electrical insulation oil, in order to decrease a competing Triphenylphosphine oxide (TPPO) formation reaction.

The system may further comprises a mixing unit for mixing highly concentrated powder based phosphine product into an oil base, prior to the addition of phosphine product to said insulation oil and an injection device comprises a dosage unit for controlling an amount of phosphine product to be added.

1. A condition diagnosis and a method for removal of elemental sulfur from electrical insulation oil in an oil filled apparatus comprising the steps of:
a)-sampling said electrical insulation oil of said oil filled apparatus;
b)-determining the presence of elemental sulfur in said electrical insulation oil;
c)-if elemental sulfur is present in said insulation oil, a sulfidation potential exists; and then
d)-slowly adding a desired amount of a phosphine product to said electrical insulation oil.

2. The method according to claim 1, wherein step b) comprises a concentration level determination of elemental sulphur.

3. The method according to claim 2, wherein a desired amount of phosphine product corresponds to at least a stoichiometric amount of phosphine related to said elemental sulfur in said insulation oil.

4. The method according to claim 1, wherein Tri-Phenyl-Phosphine (TPP) is used as said phosphine product.

5. The method according to claim 1, further comprising, before adding said phosphine product, removing gases present in said electrical insulation oil in order to decrease a competing Triphenylphosphine oxide (TPPO) formation reaction.

6. The method according to claim 1, wherein before the addition of phosphine product to said oil:
providing a highly concentrated powder based phosphine product; and
adding said highly concentrated powder phosphine product in an oil base.

7. The method according to claim 1, further comprising the step of adding said phosphine product as a concentrated hydrocarbon based solution.

8. The method according to claim 1, further comprising the steps of:
e) sampling from said electrical insulation oil; and
f) determining obtained TPP and TPPS concentrations.

9. The method according to claim 1, further comprising the steps of:
sampling from said electrical insulation oil; and
repeating step b), on a periodic basis, after addition of said phosphine product to determine TPP, TPPS and TPPO.

10. The method according to claim 8, based on said determined TPP, repeating the method from step c) upon desire.

11. The method according to claim 1, wherein step a) and b) are performed on-line.

12. The method according to claim 1, wherein all steps are performed on site.

13. The method according to claims 12, wherein step b) is performed at a laboratory off site.

14. A system for condition diagnosis and removal of elemental sulfur from electrical insulation oil in an oil filled apparatus comprising
a)-a circulation circuit for said electrical insulation oil;
b)-a sampler, at a sampler point, in fluid communication to said electrical insulation oil circuit for sampling said electrical insulation oil of said oil filled apparatus;
c)-an analyzing apparatus for determining concentration of elemental sulfur in said electrical insulation oil; and
d) an injection device, at an injection point, in fluid communication for slowly injection of a desired amount of a phosphine product to said electrical insulation oil.

15. The system according to claim 14, wherein the system further comprises a degasser in fluid communication with said electrical insulation oil circuit, for removal of gases present in said electrical insulation oil, in order to decrease a competing Triphenylphosphine oxide (TPPO) formation reaction.

16. The system according to claim 14, wherein the system further comprises a mixing unit for mixing highly concentrated powder based phosphine product into an oil base, prior to the addition of phosphine product to said insulation oil.

17. The system according to claim 14, wherein said injection device is an oil pump.

18. The system according to claim 14, wherein said injection device comprises a dosage unit for controlling an amount of phosphine product to be added.

19. The system according to claim 14, wherein said analyzing apparatus is arranged for determining obtained TPP and TPPS concentrations.

20. The system according to claim 14, wherein said analyzing apparatus is a gas chromatograph.
